(19) United States
(12) Patent Application Publication
(10) Pub. No.: US 2024/0225624 A1
(43) Pub. Date: Jul. 11, 2024

(54) TISSUE SAMPLE DEVICE AND METHODS

(71) Applicant: Boston Scientific Scimed, Inc., Maple Grove, MN (US)

(72) Inventors: Andrew James Schaubhut, Bolton, MA (US); Peter L. Dayton, Brookline, MA (US); James Weldon, Newton, MA (US); Paul Smith, Smithfield, RI (US); Madinah Shamsu, Antioch, TN (US)

(73) Assignee: Boston Scientific Scimed, Inc., Maple Grove, MN (US)

(21) Appl. No.: 18/410,459

(22) Filed: Jan. 11, 2024

Related U.S. Application Data
(60) Provisional application No. 63/438,377, filed on Jan. 11, 2023.

(51) Int. Cl. A61B 10/04; A61B 10/02

(52) U.S. Cl. CPC A61B 10/04; A61B 10/0233; A61B 2017/2212

(57) ABSTRACT

A tissue collection system for collection a sample from the body. The system may include an outer tubular member having a proximal end region and a distal end region and defining a lumen extending from the proximal end region to the distal end region, an inner tubular member slidably disposed within the lumen of the outer tubular member, the inner tubular member defining a lumen extending from a proximal end region to a distal end region of the inner tubular member and an expandable tissue collection device disposed adjacent to the distal end region of the inner tubular member.

### CROSS-REFERENCE TO RELATED APPLICATIONS

This application claims the benefit of priority under 35 U.S.C. § 119 of U.S. Provisional Application No. 63/438,377, filed Jan. 11, 2023, the entire disclosure of which is hereby incorporated by reference.

### TECHNOLOGY FILED

The present application relates generally to medical devices for tissue sample collection, and more particularly, to medical devices for tissue sample collection resulting in greater cellular or other tissue yield.

### BACKGROUND

Certain medical tests require sampling of cells from target areas of a subject's body. For instance, a screening test for detecting potentially pre-cancerous and cancerous tissues in a subject's body may include taking samples of tissue or cells from a target area of the subject's body. A tissue collection device may be used to collect cells or other tissues from the target area. Tissue collection from some parts of the anatomy may be difficult.

There is an ongoing need to provide alternative medical devices as well as alternative methods for manufacturing and using medical devices.

### SUMMARY

This disclosure provides design, material, manufacturing methods, and use alternatives for medical devices.

In a first example, a tissue collection system may comprise an outer tubular member having a proximal end region and a distal end region and defining a lumen extending from the proximal end region to the distal end region, an inner tubular member slidably disposed within the lumen of the outer tubular member, the inner tubular member defining a lumen extending from a proximal end region to a distal end region of the inner tubular member, and a tissue collection device disposed adjacent to the distal end region of the inner tubular member, the tissue collection device comprising an expandable basket. The tissue collection device may be movable between a retracted delivery position and an extended sample collection position.

Alternatively or additionally to any of the examples above, in another example, the expandable basket may comprise a plurality of longitudinally extending struts.

Alternatively or additionally to any of the examples above, in another example, one or more edges of at least one strut of the plurality of struts may have a sharpened surface.

Alternatively or additionally to any of the examples above, in another example, a surface of at least one strut of the plurality of struts may be textured.

Alternatively or additionally to any of the examples above, in another example, the tissue collection system may further comprise one or more barbs coupled to at least one strut of the plurality of struts.

Alternatively or additionally to any of the examples above, in another example, the one or more barbs may comprise a wire wound around the at least one strut.

Alternatively or additionally to any of the examples above, in another example, at least one free end of the wire may extend radially from the at least one strut.

Alternatively or additionally to any of the examples above, in another example, the tissue collection system may further comprise one or more teeth extending from at least one strut.

Alternatively or additionally to any of the examples above, in another example, the one or more teeth may be formed as a single monolithic structure with the at least one strut.

Alternatively or additionally to any of the examples above, in another example, the plurality of struts may comprise a plurality of individual filaments, the filaments coupled or connected to one another at their respective proximal and distal ends.

Alternatively or additionally to any of the examples above, in another example, the tissue collection device may comprise a cut tube.

Alternatively or additionally to any of the examples above, in another example, the tissue collection device may be a monolithic structure with the inner tubular member.

Alternatively or additionally to any of the examples above, in another example, the tissue collection device may be coupled to the inner tubular member.

Alternatively or additionally to any of the examples above, in another example, the tissue collection device may be self-expanding.

Alternatively or additionally to any of the examples above, in another example, the tissue collection system may further comprise an actuation mechanism coupled to a proximal or distal end of the tissue collection device.

Alternatively or additionally to any of the examples above, in another example, the inner tubular member may further comprise one or more cut-out regions, the one or more cut-out regions may be adjacent to the tissue collection device.

Alternatively or additionally to any of the examples above, in another example, the plurality of struts may have a generally flattened ribbon-like shape.

Alternatively or additionally to any of the examples above, in another example, the plurality of struts may have a generally wire-like shape.

Alternatively or additionally to any of the examples above, in another example, a proximal end of each strut of the plurality of struts may be coupled to a proximal collar and a distal end of each strut of the plurality of struts may be coupled to a distal collar.

Alternatively or additionally to any of the examples above, in another example, at least one of the proximal collar or the distal collar may be movably disposed over the inner tubular member.

Alternatively or additionally to any of the examples above, in another example, in an expanded configuration, an intermediate region of each strut of the plurality of struts may flex radially outwards.

Alternatively or additionally to any of the examples above, in another example, each strut of the plurality of struts each may include a proximal end region and a distal end region.

Alternatively or additionally to any of the examples above, in another example, the proximal end regions of the plurality of struts may meet the distal end regions at an intersection point.

Alternatively or additionally to any of the examples above, in another example, in an expanded configuration, the proximal end regions of the plurality of struts may extend at a first angle relative to a longitudinal of the expandable basket and the distal end regions of the plurality of struts may extend at a second angle relative to a longitudinal of the expandable basket.

Alternatively or additionally to any of the examples above, in another example, the first angle may be different from the second angle.

Alternatively or additionally to any of the examples above, in another example, in an expanded configuration the struts may flex radially outwards such that a proximal end region and a distal end region of the expandable basket may extend at a non-parallel angle relative to a longitudinal axis of the expandable basket and an intermediate region may extend approximately parallel to the longitudinal axis of the expandable basket.

In another example, a tissue collection system may comprise an outer tubular member having a proximal end region and a distal end region and defining a lumen extending from the proximal end region to the distal end region, an inner tubular member slidably disposed within the lumen of the outer tubular member, the inner tubular member defining a lumen extending from a proximal end region to a distal end region of the inner tubular member, and a tissue collection device disposed adjacent to the distal end region of the inner tubular member, the tissue collection device comprising a radially expandable basket having a plurality of longitudinally extending struts extending between a proximal collar and a distal collar. The tissue collection device may be movable between a retracted collapsed delivery position and an extended expanded sample collection position.

Alternatively or additionally to any of the examples above, in another example, the tissue collection device may be a cut tube.

Alternatively or additionally to any of the examples above, in another example, at least one strut of the plurality of struts may include a radially extending tissue disruption feature.

In another example, a tissue collection system may comprise an outer tubular member having a proximal end region and a distal end region and defining a lumen extending from the proximal end region to the distal end region, an inner tubular member slidably disposed within the lumen of the outer tubular member, the inner tubular member defining a lumen extending from a proximal end region to a distal end region of the inner tubular member, a tissue collection device disposed adjacent to the distal end region of the inner tubular member, the tissue collection device comprising a radially expandable basket having a plurality of longitudinally extending wires extending between a proximal collar and a distal collar, and at least one barb coupled to at least one wire of the plurality of longitudinally extending wires, the at least one barb including a radially extending free end. The tissue collection device may be movable between a retracted collapsed delivery position and an extended expanded sample collection position.

The above summary of some example embodiments is not intended to describe each disclosed embodiment or every implementation of the disclosure.

### BRIEF DESCRIPTION OF THE DRAWINGS

The disclosure may be more completely understood in consideration of the following detailed description of various embodiments in connection with the accompanying drawings, in which:

FIG. 1 illustrates a partial cross-sectional side view of an illustrative tissue collection device system for delivering a tissue collection device to a target region in a retracted or delivery configuration;

FIG. 2 illustrates a perspective view of the illustrative tissue collection device of FIG. 1;

FIG. 3 illustrates a perspective view of another illustrative tissue collection device;

FIG. 4 illustrates a side view of another illustrative tissue collection device system for delivering a tissue collection device to a target region in a deployed configuration; and

FIG. 5 is an illustrative flow chart of a method for collecting a tissue sample using the systems of FIGS. 1-4.

While the disclosure is amenable to various modifications and alternative forms, specifics thereof have been shown by way of example in the drawings and will be described in detail. It should be understood, however, that the intention is not to limit aspects of the disclosure to the particular embodiments described. On the contrary, the intention is to cover all modifications, equivalents, and alternatives falling within the spirit and scope of the disclosure.

### DETAILED DESCRIPTION OF EXEMPLARY EMBODIMENTS

All numeric values are herein assumed to be modified by the term “about”, whether or not explicitly indicated. The term “about” generally refers to a range of numbers that one of skill in the art would consider equivalent to the recited value (i.e., having the same function or result). In many instances, the term “about” may be indicative as including numbers that are rounded to the nearest significant figure.

The recitation of numerical ranges by endpoints includes all numbers within that range (e.g., 1 to 5 includes 1, 1.5, 2, 2.75, 3, 3.80, 4, and 5).

Although some suitable dimensions, ranges, and/or values pertaining to various components, features, and/or specifications are disclosed, one of skill in the art, incited by the present disclosure, would understand desired dimensions, ranges, and/or values may deviate from those expressly disclosed.

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise.

The term “distal” refers to a portion farthest away from a user when introducing a device into a patient. By contrast, the term “proximal” refers to a portion closest to the user when placing the device into the patient. As used herein, the terms “comprises,” “comprising,” or any other variation thereof, are intended to cover a non-exclusive inclusion, such that a process, method, article, or apparatus that comprises a list of elements does not necessarily include only those elements, but may include other elements not expressly listed or inherent to such process, method, article, or apparatus. Additionally, terms that indicate the geometric shape of a component/surface refer to exact and approximate shapes.

The following detailed description should be read with reference to the drawings in which similar elements in different drawings are numbered the same. The detailed description and the drawings, which are not necessarily to scale, depict illustrative embodiments and are not intended to limit the scope of the disclosure. The illustrative embodiments depicted are intended only as exemplary. Selected features of any illustrative embodiment may be incorporated into an additional embodiment unless clearly stated to the contrary.

Endoscopic retrograde cholangiopancreatography (ERCP) is a procedure that utilizes both endoscopic and fluoroscopic techniques to diagnose and treat issues arising in the common bile duct (CBD) and pancreatic ducts (PD). One of the main issues being treated today is strictures in the CBD from such as, but not limited to, primary sclerosing cholangitis (PSC), cancer of the bile duct, damage and scarring due to a gallstone in the bile duct, etc. However, biliary structures may have a low cancer sensitivity rate which may lead to a false-negative diagnosis during an ERCP procedure. In some cases, low sensitivity may be linked to inadequate tissue sampling which is a primary limiting factor of detecting potential malignancy. When a clinician needs to take a sample of the stricture, one of the most common ways to do this is by using a cytology brush. However, the sensitivity of a cytology brush may be between about 30-60%. The sensitivity of cytology brushes may be mainly linked to insufficient quantities of samples. One of the reasons cytology brushes may not be able to collect enough samples is the use of soft bristles. Another reason may be losing samples during the procedure.

What may be desirable is a sample collection device that collects a sufficient amount of samples for better prediction of biliary cancer. While the present disclosure is described with respect to the common bile duct and pancreatic ducts, the devices and methods are not limited to such use. For example, the devices and methods described herein may be used in any portion of the anatomy, as desired. Further, the devices and methods described herein may be used either endoscopic or non-endoscopic anatomies. Some illustrative anatomies may include, but are not limited to, the mouth, the esophagus, the stomach, the duodenum, other portions of the gastrointestinal tract, the pathways leading to the lungs, other portions of the respiratory system, the urinary tract, the cervix, other reproductive anatomy, etc.

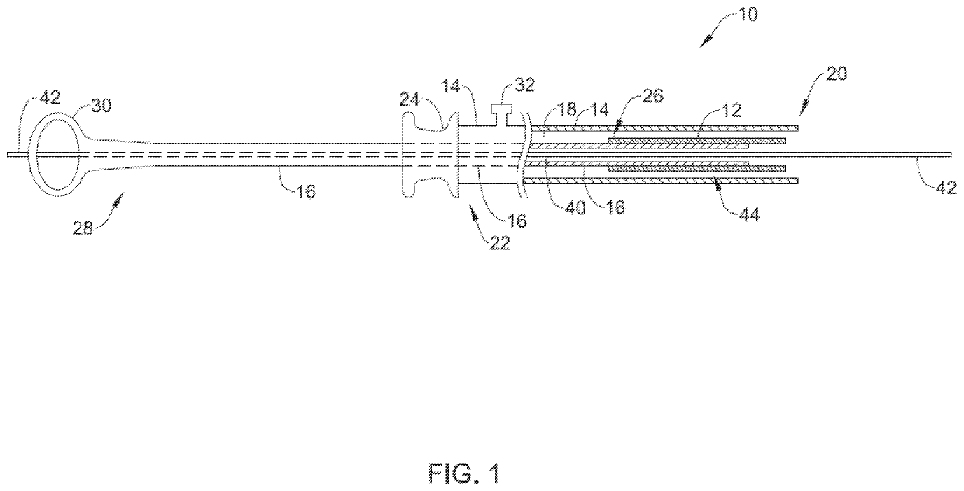

FIG. 1 is a partial cross-sectional side view of an illustrative tissue collection device system 10 for delivering a tissue collection device 12 to a target region, such as, but not limited to, the common bile duct or pancreatic duct, in a retracted or delivery configuration. The tissue collection system 10 may include an outer or exterior elongate shaft or tubular member 14 and an inner elongate shaft or tubular member 16. The inner tubular member 16 may be slidably disposed within a lumen 18 of the outer tubular member 14. The outer tubular member 14 may extend proximally from a distal end region 20 to a proximal end region 22 configured to remain outside of a patient's body. A first hub or handle 24 may be coupled to the proximal end region 22 of the outer tubular member 14. In some cases, a port 32, such as an injection port, may be provided in the outer tubular member 14. Other structures to facilitate connection to other medical devices (e.g., syringe, stopcocks, Y-adapter, etc.) and to provide access to lumen 18 may be provided. The inner tubular member 16 may extend proximally from a distal end region 26 to a proximal end region 28 configured to remain outside of a patient's body. A second hub or handle 30 may be coupled to the proximal end region 28 of the inner tubular member 16.

The outer tubular member 14 may include a lumen 18 extending from the distal end region 20 to the proximal end region 22. The lumen 18 may also extend through the first handle 24. The lumen 18 of the outer tubular member 14 and the first handle 24 may be configured to slidably receive the inner tubular member 16. The inner tubular member 16 may include a lumen 40 extending from the distal end region 26 to the proximal end region 28. The lumen 40 of the inner tubular member 16 may also extend through the second handle 30. The lumen 40 of the inner tubular member 16 may be configured to receive a guidewire 42, as desired. In other examples, the guidewire 42 may be received within the lumen 18 of the outer tubular member 14. It is contemplated that the system 10 may be arranged such that the guidewire 42 extends within a lumen along an entire length of the outer tubular member 14 or the inner tubular member 16 in an “over-the-wire” manner or the guidewire 42 may exit a side port in the outer tubular member 14 distal to the proximal end region 22 thereof in a “rapid-exchange” manner.

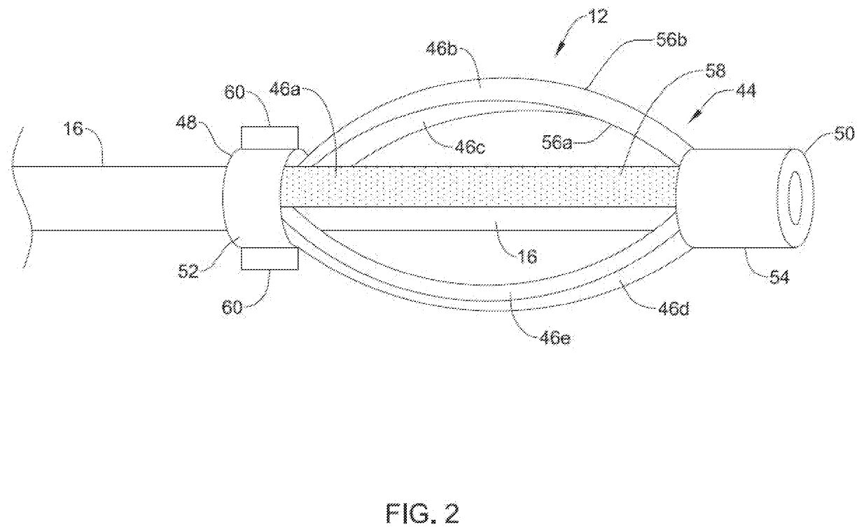

The tissue collection device 12 may be disposed around or formed from a portion of the inner tubular member 16 at or adjacent to the distal end region 26 thereof. The tissue collection device 12 may extend about an entirety of the circumference of the inner tubular member 16. In other embodiments, the tissue collection device 12 may be radially spaced about a circumference of the inner tubular member 16 in a uniform pattern or eccentric manner, as desired. In some embodiments, the tissue collection device 12 may include a radially expanding frame or basket 44 moveable between a collapsed delivery configuration (FIG. 1) and an expanded use configuration (FIG. 2). While the tissue collection device 12 is described as an expandable basket, it is contemplated that other tissue collection devices may be used, as desired.

Referring additionally to FIG. 2, which illustrates a perspective view of the illustrative tissue collection device 12, the expandable basket 44 may extend from a proximal end 48 to a distal end 50 and include a plurality of longitudinally extending struts 46a-e (collectively, 46). In some embodiments, the proximal end 48 of the basket 44 may extend proximally to a location configured to remain outside the body such that the tissue collection device 12 may be manipulated through actuation of the proximal end 48, although this is not required. The expandable basket 44 may include any number of struts 46 desired, such as, but not limited to, one, two, three, four, five, six, or more. The struts 46 may have a generally flattened ribbon-like shape (e.g., having a width greater than a thickness). In other examples, the struts 46 may have a generally wire-like shape (e.g., having a similar width and thickness). However, the struts 46 may take any shape desired. The struts 46 may be uniformly spaced about a circumference of the expandable basket 44. However, this is not required. In some cases, the struts 46 may be eccentrically spaced.

The proximal ends of the struts 46 may be secured to a proximal collar 52 and the distal ends of the struts 46 may be secured to a distal collar 54. In some embodiments, the struts 46 may be formed as a single monolithic structure with the proximal collar 52 and/or the distal collar 54. For example, the struts 46 and collars 52, 54 may be cut from a single tube. It is further contemplated that the struts 46 and collars 52, 54 may be formed from or as a part of the inner tubular member 16. Said differently, the inner tubular member 16, struts 46, and collars 52, 54 may be formed as a single monolithic structure. For example, the cut tube may be the inner tubular member 16 or a separate tube, as desired. In other embodiments, the struts 46 and one or more of the proximal and distal collars 52, 54 may be formed as separate structures that are coupled together. For example, the struts 46 may be welded, brazed, soldered, adhered, etc. to the proximal and/or distal collars 52, 54. Other coupling techniques may be used, as desired. It is contemplated that the coupling mechanism may be dependent, at least in part, on the material of the struts 46 and/or the collars 52, 54. When the expandable basket 44 is a separate component from the inner tubular member 16, at least one of the proximal collar 52 or the distal collar 54 may be fixedly secured to an outer surface of the inner tubular member 16 while the other of the proximal collar 52 or the distal collar 54 is movably disposed over the inner tubular member 16 to allow the basket 44 to radially expand. In one example, the proximal collar 52 may be fixedly secured to the inner tubular member 16 while the distal collar 54 may be slidably disposed over the inner tubular member 16. In such a configuration, the distal collar 54 may move distally to collapse the expandable basket 44 and move proximally to expand the expandable basket 44. The reverse configuration is also contemplated in which the distal collar 54 may be fixedly secured to the inner tubular member 16 while the proximal collar 52 may be slidably disposed over the inner tubular member 16.

The expandable basket 44 may be self-expandable or may require an external force to expand from a collapsed state. Self-expandable members may be formed of any material or structure that is in a compressed state when force is applied and in an expanded state when force is released. Such members may be formed, for example, of shape memory alloys such as nitinol or any other self-expandable materials. When employing such shape-memory materials, the expandable basket 44 may be heat set in the expanded state and then compressed to fit within the outer tubular member 14, for example. In another embodiment, a spring may be provided to effect expansion. It is contemplated that nickel-titanium alloys may enable kink-resistant folding and self-expansion. In other examples, magnetic alloys, metals, metal alloys, polymers, composites, etc. may be used to form the expandable basket 44.

In other instances, a manual force applied to the inner tubular member 16 may manipulate or actuate the expandable basket 44 between the expanded and collapsed state. For example, an actuation element may include a central wire or rod that extends through the expandable basket 44 and is coupled to the distal collar 54. Alternatively, external forces such as, but not limited to, pneumatic methods, compressed fluid, pull wires, push wires, rods, or the like may also be employed to expand the expandable basket 44. According to this embodiment, a pulling force exerted proximally on the wire may allow the struts 46 to expand and move the expandable basket 44 into an expanded state. A pushing force exerted distally on the wire may move elongate the struts 46 and/or otherwise shift the expandable basket 44 to a compressed or elongated state. Other actuation mechanisms may also be utilized. For example, the guidewire 42 may be used to exert a proximal pushing force on a distal end of the distal collar 52. In such an instance, the guidewire 42 may include an enlarged region having a diameter greater than a diameter of the lumen of the tissue collection device 12 such that proximal actuation of the guidewire 42 causes the enlarged region of the guidewire 42 to contact the distal collar 52 and further proximal actuation of the guidewire 42 causes the distal collar 52 to move proximally and expand the expandable basket 44. It is contemplated that manual actuation of the expandable basket 44 using a wire, rod, etc., may put a higher force on the expandable basket 44 which may allow for greater engagement of the basket with a target tissue.

When the tissue collection device 12 is disposed within the outer tubular member 14, the expandable basket 44 may be restrained in a compressed reduced diameter or delivery configuration by the outer tubular member 14 surrounding the tissue collection device 12. In other examples, the expandable basket 44 may assume a collapsed configuration until an actuation force is applied to the expandable basket 44. In the collapsed configuration, the tissue collection device 12 may have a smaller diameter than the expanded deployed configuration. The distal end region 20 of the outer tubular member 14 may be positioned such that the outer tubular member 14 surrounds and covers the length of the tissue collection device 12 during delivery. The outer tubular member 14 may have sufficient hoop strength to retain the tissue collection device 12 in its reduced diameter state. In the expanded configuration, an intermediate region of the struts 46 may flex radially outward such that the struts 46 from an arc or a curved configuration.

The tissue collection system 10 may be advanced through the body towards the target location, as desired. The tissue collection system 10 may be advanced with or without the use of a guidewire 42. Once the tissue collection device 12 is positioned adjacent to the target region, the restraining forces maintaining the tissue collection device 12 in the radially compressed configuration may be removed to deploy the tissue collection device 12.

The tissue collection device 12 may be deployed by actuating the second handle 30, for example, by distally pushing the second handle 30, while maintaining the first handle 24 in a fixed position. Thus, the inner tubular member 16 may be distally advanced relative to the outer tubular member 14. In other words, the inner tubular member 16 may be distally advanced while the outer tubular member 14 is held stationary. The reverse configuration is also contemplated. For example, the outer tubular member 14 may be proximally retracted while the inner tubular member 16 is held stationary. As the inner tubular member 16 is distally advanced, the biasing force is removed from the exterior of the tissue collection device 12 and the expandable basket 44 may assume its radially expanded, unbiased, deployed configuration, shown in FIG. 2. Alternatively, the expandable basket 44 may remain collapsed until an actuation force is applied to the expandable basket 44 (e.g., using a pull-wire or other actuation mechanism) to move the expandable basket 44 to the radially expanded, deployed configuration.

In the radially expanded configuration, the struts 46 may bow radially outwards such that the expandable basket 44 has a generally ovoid shape. However, the expandable basket 44 may take other shapes in the expanded configuration, as desired. The expandable basket 44 may be sized and shaped such that when the expandable basket 44 is in the expanded configuration, the struts 46 may interface with a collection site. In some cases, the expandable basket 44 may be expanded until at least a portion of the struts 46 contact the collection site. It is contemplated that in some cases, the expandable basket 44 may not fully expand before contacting the collection site.

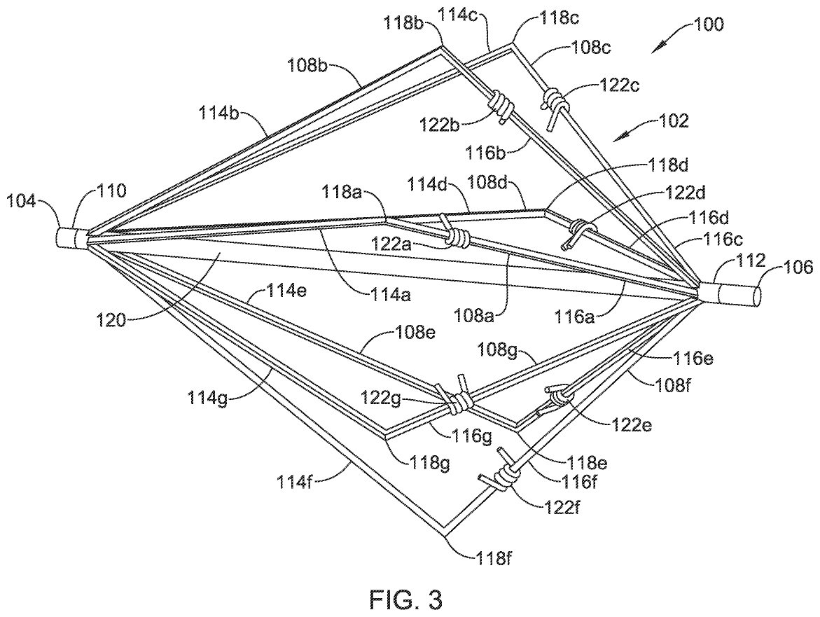

Once the tissue collection device 12 is deployed from the outer tubular member 14 and the expandable basket 44 is expanded, the second handle 30 may be actuated to repeatedly distally advance, proximally retract, and/or rotate the tissue collection device 12 along the target collection site. This may cause the struts 46 to brush against the tissue surface to dislodge and capture cells. It is contemplated that the edges 56a, 56b of the struts 46 may be angled or sharpened to scratch the collection site. In the illustrated embodiment, each edge of the plurality of struts 46 is not labeled for brevity and ease of understanding. In some examples, the struts 46 may further include texturing 58, such as, but not limited to, a roughed or grainy surface, on an outer surface and/or inner surface thereof. The texturing may be configured to release and capture cells from the stricture. The texturing 58 may be macro-texturing or micro-texturing, as desired. In some examples, the texturing 58 may include holes or modified holes having an opening that partially extends radially from a surface of the struts 46 such that an edge of the modified hold scrapes tissue into the hole. It yet other examples, the struts 46 may include barbs. The barbs may extend radially inward or radially outwards from the struts 46 and may be configured to further disrupt the collection site to release cells for collection. The barbs may be similar in form and function to the barbs 122 shown and described with respect to FIG. 3. It is further contemplated that one or more of the struts 46 may include other tissue engaging features, such as, but not limited to, teeth or bristles, anywhere along a length of the struts 46.

The tissue collection device 12 may further include one or more optional tissue capturing mechanisms 60. The tissue capturing mechanism 60 may include a brush or membrane that may collect or trap cells that have been dislodged by the expandable basket 44. Some illustrative brushes are described in commonly assigned U.S. Patent Application No. 63/309,818, titled TISSUE SAMPLE DEVICE AND METHODS, the disclosure of which is hereby incorporated by reference. It is contemplated that the tissue capturing mechanism 60 may be positioned proximal to the expandable basket, distal to the expandable basket 44, or anywhere along a length of the expandable basket 44, as desired.

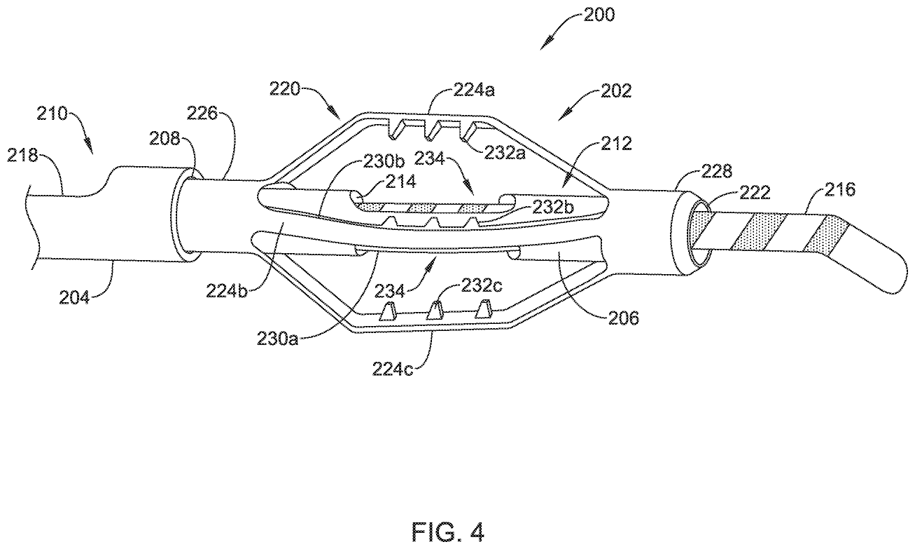

Once the clinician has captured cells from the target site, the inner tubular member 16 may be proximally retracted until the tissue collection device 12 is disposed within the lumen 18 of the outer tubular member 14. When the expandable basket 44 is self-expanding, the proximal retraction of the inner tubular member 16 may cause the basket 44 to collapse as the basket 44 is drawn into the lumen 18 of the outer tubular member 14. When expandable basket 44 is manually expanded, the basket 44 may be moved into the collapsed configuration before proximally retracting the inner tubular member 16. In some embodiments, the inner tubular member 16 may include one or windows or cut-out regions for trapping cells or tissues therein as the expandable basket 44 is collapsed. The windows may be similar in form and function to the cut-out regions 234 described with respect to FIG. 4.

FIG. 3 illustrates a perspective view of another illustrative tissue collection device 100 including an expandable basket 102, in an expanded configuration, that may be used with the system 10 of FIG. 1. The expandable basket 102 may extend from a proximal end 104 to a distal end 106 and include a plurality of longitudinally extending struts 108a-g (collectively, 108). In some embodiments, the proximal end 104 of the basket 102 may extend proximally to a location configured to remain outside the body such that the tissue collection device 100 may be manipulated through actuation of the proximal end 104, although this is not required. The expandable basket 102 may include any number of struts 108 desired, such as, but not limited to, one, two, three, four, five, six, seven, or more. The individual struts 108 may be formed from a wire or filament. While the struts 108 are described as being formed from a wire, the struts 108 may have any number of cross-sectional shapes including, but not limited to, circular, square, rectangular, polygonal, elliptical, oblong, etc. In other examples, the struts 108 may have a generally flat ribbon-like shape (e.g., having a width greater than a thickness). However, the struts 108 may take any shape desired. The struts 108 may be uniformly spaced about a circumference of the expandable basket 102. However, this is not required. In some cases, the struts 108 may be eccentrically spaced. The expandable basket 102 may include one or more radiopaque markers positioned anywhere along a length thereof for allowing a location of the expandable basket 102 to be vied on a fluoroscopy screen or another imaging technique.

The proximal ends of the struts 108 may be secured to a proximal collar 110 and the distal ends of the struts 108 may be secured to a distal collar 112. In some embodiments, the struts 108 may be formed a single monolithic structure with the proximal collar 110 and/or the distal collar 112. For example, the struts 108 and collars 110, 112 may be cut from a single tube. It is further contemplated that the struts 108 and collars 110, 112 may be formed from the inner tubular member 16. For example, the cut tube may be the inner tubular member 16 or a separate tube, as desired. In other embodiments, the struts 108 and one or more of the proximal and distal collars 110, 112 may be formed as separate structures that are coupled together. For example, the struts 108 may be welded, brazed, soldered, adhered, etc. to the proximal and/or distal collars 110, 112. Other coupling techniques may be used, as desired. It is contemplated that the coupling mechanism may be depend, at least in part, on the material of the struts 108 and/or the collars 110, 112. When the expandable basket 102 is a separate component from the inner tubular member 16, at least one of the proximal collar 110 or the distal collar 112 may be fixedly secured to an outer surface of the inner tubular member 16 while the other of the proximal collar 110 or the distal collar 112 is movably disposed over the inner tubular member 16 to allow the basket 102 to radially expand. In one example, the proximal collar 110 may be fixedly secured to the inner tubular member 16 while the distal collar 112 may be slidably disposed over the inner tubular member 16. In such a configuration, the distal collar 112 may move distally to collapse the expandable basket 102 and move proximally to expand the expandable basket 102. The reverse configuration is also contemplated in which the distal collar 112 may be fixedly secured to the inner tubular member 16 while the proximal collar 110 may be slidably disposed over the inner tubular member 16. Alternatively, the proximal collar 110 and/or distal collar 112 may be coupled to a centrally extending tubular member 120. The tubular member 120 may be used in in place of or in addition to the inner tubular member 16.

The struts 108 may each include a proximal end region 114a-g (collectively, 114) and a distal end region 116a-g (collectively, 116). The proximal end regions 114 may meet the distal end regions 116 at an intersection point 118a-g. In the expanded configuration, the proximal end regions 114 of the struts 108 may extend at a first angle relative to a longitudinal of the expandable basket 102 and the distal end regions 116 may extend at a second angle relative to a longitudinal of the expandable basket 102. The first angle may be different from the second angle.

The struts 108 may each include one or more barbs 122a-g (collectively, 122) wrapped about the struts 108. While each strut 108 is shown as including a single barb 122, it is contemplated each strut 108 may have more than one barb 122. It is further contemplated that some struts 108 may be free from a barb 122. The struts 108 may each have a same number of barbs 122 or a differing number of barbs 122, as desired. In the illustrated embodiment, the barbs 122 may be a wire wound or wrapped about the struts 108 with the free ends extending radially away from the struts 108. The free ends may extend radially inward, radially outward, or combinations thereof, as desired. It is contemplated that the barbs 122 may be configured to disrupt and trap tissue from a target collection site.

The expandable basket 102 may be self-expandable or may require an external force to expand from a collapsed state. Self-expandable members may be formed of any material or structure that is in a compressed state when force is applied and in an expanded state when force is released. Such members may be formed, for example, of shape memory alloys such as nitinol or any other self-expandable materials. When employing such shape-memory materials, the expandable basket 102 may be heat set in the expanded state and then compressed to fit within the outer tubular member 14, for example. In another embodiment, a spring may be provided to effect expansion. It is contemplated that nickel-titanium alloys may enable kink-resistant folding and self-expansion. In other examples, magnetic alloys, metals, metal alloys, polymers, composites, etc. may be used to form the expandable basket 102.

In other instances, a manual force applied to the inner tubular member 16 may manipulate or actuate the expandable basket 102 between the expanded and collapsed state. For example, an actuation element may include a central wire or rod that extends through the expandable basket 102 and is coupled to the distal collar 112. Alternatively, external forces such as, but not limited to, pneumatic methods, compressed fluid, pull wires, push wires, rods, or the like may also be employed to expand the expandable basket 102. According to this embodiment, a pulling force exerted proximally on the wire may allow the struts 108 to expand and move the expandable basket 102 into an expanded state. A pushing force exerted distally on the wire may move elongate the struts 108 and/or otherwise shift the expandable basket 102 to a compressed or elongated state. Other actuation mechanisms may also be utilized. For example, the guidewire 42 may be used to exert a proximal pushing force on a distal end of the distal collar 110. In such an instance, the guidewire 42 may include an enlarged region having a diameter greater than a diameter of the lumen of the tissue collection device 100 such that proximal actuation of the guidewire 42 causes the enlarged region of the guidewire 42 to contact the distal collar 110 and further proximal actuation of the guidewire 42 causes the distal collar 110 to move proximally and expand the expandable basket 102. It is contemplated that manual actuation of the expandable basket 102 using a wire, rod, etc., may put a higher force on the expandable basket 102 which may allow for greater engagement of the basket with a target tissue.

When the tissue collection device 100 is disposed within the outer tubular member 14, the expandable basket 102 may be restrained in a compressed reduced diameter or delivery configuration by the outer tubular member 14 surrounding the tissue collection device 100. In other examples, the expandable basket 102 may assume a collapsed configuration until an actuation force is applied to the expandable basket 102. In the collapsed configuration, the tissue collection device 100 typically has a smaller diameter than the expanded deployed configuration. The distal end region 20 of the outer tubular member 14 may be positioned such that the outer tubular member 14 surrounds and covers the length of the tissue collection device 100 during delivery. The outer tubular member 14 may have sufficient hoop strength to retain the tissue collection device 100 in its reduced diameter state.

The expandable basket 102 may be deployed in a manner similar to the expandable basket 44 described herein. In the radially expanded configuration, the struts 108 may bend at the intersection point 118 to radially expand the expandable basket 102. The expandable basket 102 may be sized and shaped such that when the expandable basket 102 is in the expanded configuration, the struts 108 may interface with a collection site. In some cases, the expandable basket 102 may be expanded until at least a portion of the struts 108 contact the collection site. It is contemplated that in some cases, the expandable basket 102 may not fully expand before contacting the collection site.

Once the tissue collection device 100 is deployed from the outer tubular member 14 and the expandable basket 102 is expanded, the second handle 30 may be actuated to repeatedly distally advance, proximally retract, and/or rotate the tissue collection device 100 along the target collection site. This may cause the struts 108 to brush against the tissue surface to dislodge and capture cells. It is contemplated that the edges of the struts 108 may be angled or sharpened to scratch the collection site. In the illustrated embodiment, each edge of the plurality of struts 108 is not labeled for brevity and ease of understanding. In some examples, the struts 108 may further include texturing, such as, but not limited to, a roughed or grainy surface, on an outer surface and/or inner surface thereof. The texturing may be configured to release and capture cells from the stricture. The texturing may be macro-texturing or micro-texturing, as desired. It is further contemplated that one or more of the struts 108 may include other tissue engaging features, such as, but not limited to, teeth or bristles, anywhere along a length of the struts 108.

The tissue collection device 100 may further include one or more optional tissue capturing mechanisms (not explicitly shown). The tissue capturing mechanism may include a brush or membrane that may collect or trap cells that have been dislodged by the expandable basket 102. Some illustrative brushes are described in commonly assigned U.S. Patent Application No. 63/309,818, titled TISSUE SAMPLE DEVICE AND METHODS, the disclosure of which is hereby incorporated by reference. It is contemplated that the tissue capturing mechanism may be positioned proximal to the expandable basket, distal to the expandable basket 102, or anywhere along a length of the expandable basket 102, as desired.

Once the clinician has captured cells from the target site, the inner tubular member 16 may be proximally retracted until the tissue collection device 100 is disposed within the lumen 18 of the outer tubular member 14. When the expandable basket 102 is self-expanding, the proximal retraction of the inner tubular member 16 may cause the basket 102 to collapse as the basket 102 is drawn into the lumen 18 of the outer tubular member 14. When expandable basket 102 is manually expanded, the basket 102 may be moved into the collapsed configuration before proximally retracting the inner tubular member 16. In some embodiments, the inner tubular member 16 and/or the central tubular member 120 may include one or windows or cut-out regions for trapping cells or tissues therein as the expandable basket 102 is collapsed. The windows may be similar in form and function to the cut-out regions 234 described with respect to FIG. 4.

FIG. 4 is a side view of another illustrative tissue collection device system 200 for delivering a tissue collection device 202 to a target region, such as, but not limited to, the common bile duct or pancreatic duct, in a retracted or delivery configuration. The tissue collection system 200 may include an outer or exterior elongate shaft or tubular member 204 and an inner elongate shaft or tubular member 206. The inner tubular member 206 may be slidably disposed within a lumen 208 of the outer tubular member 204. The outer tubular member 204 may extend proximally from a distal end region 210 to a proximal end region (not explicitly shown) configured to remain outside of a patient's body. A first hub or handle, similar in form and function to handle 24 described herein, may be coupled to the proximal end region of the outer tubular member 204. In some cases, a port, such as an injection port, may be provided in the outer tubular member 204. Other structures to facilitate connection to other medical devices (e.g., syringe, stopcocks, Y-adapter, etc.) and to provide access to lumen 208 may be provided. The inner tubular member 206 may extend proximally from a distal end region 212 to a proximal end region (not explicitly shown) configured to remain outside of a patient's body. A second hub or handle (not explicitly shown) may be coupled to the proximal end region of the inner tubular member 206.

The outer tubular member 204 may include a lumen 208 extending from the distal end region 210 to the proximal end region. The lumen 208 may also extend through the first handle. The lumen 208 of the outer tubular member 204 and the first handle may be configured to slidably receive the inner tubular member 206. The inner tubular member 206 may include a lumen 214 extending from the distal end region 212 to the proximal end region. The lumen 214 of the inner tubular member 206 may also extend through the second handle. The lumen 214 of the inner tubular member 206 may be configured to receive a guidewire 216, as desired. In other examples, the guidewire 216 may be received within the lumen 208 of the outer tubular member 204. It is contemplated that the system 200 may be arranged such that the guidewire 216 extends within a lumen along an entire length of the outer tubular member 204 or the inner tubular member 206 in an “over-the-wire” manner or the guidewire 216 may exit a side port 218 in the outer tubular member 204 distal to the proximal end region 22 thereof in a “rapid-exchange” manner.

The tissue collection device 202 may be disposed around or formed from a portion of the inner tubular member 206 at or adjacent to the distal end region 212 thereof. The tissue collection device 202 may extend about an entirety of the circumference of the inner tubular member 206. In other embodiments, the tissue collection device 202 may be radially spaced about a circumference of the inner tubular member 206 in a uniform pattern or eccentric manner, as desired. In some embodiments, the tissue collection device 202 may include a radially expanding frame or basket 220 moveable between a collapsed delivery configuration (not explicitly shown) and an expanded use configuration (FIG. 4). While the tissue collection device 202 is described as an expandable basket, it is contemplated that other tissue collection devices may be used, as desired.

The expandable basket 220 may extend from a proximal end region (not explicitly shown) to a distal end 222 and include a plurality of longitudinally extending struts 224a-c (collectively, 224). In some embodiments, the proximal end of the basket 220 may extend proximally to a location configured to remain outside the body such that the tissue collection device 202 may be manipulated through actuation of the proximal end, although this is not required. The expandable basket 220 may include any number of struts 224 desired, such as, but not limited to, one, two, three, four, five, six, or more. The struts 224 may have a generally flattened ribbon-like shape (e.g., having a width greater than a thickness). In other examples, the struts 224 may have a generally wire-like shape. However, the struts 224 may take any shape desired. The struts 224 may be uniformly spaced about a circumference of the expandable basket 220. However, this is not required. In some cases, the struts 224 may be eccentrically spaced. The expandable basket 220 may include one or more radiopaque markers positioned anywhere along a length thereof for allowing a location of the expandable basket 220 to be vied on a fluoroscopy screen or another imaging technique.

The proximal ends of the struts 224 may be secured to a proximal collar 226 and the distal ends of the struts 224 may be secured to a distal collar 228. In some embodiments, the struts 224 may be formed as a single monolithic structure with the proximal collar 226 and/or the distal collar 228. For example, the struts 224 and collars 226, 228 may be cut from a single tube. It is further contemplated that while not explicitly shown, the struts 224 and collars 226, 228 may be formed from the inner tubular member 206. For example, the cut tube may be the inner tubular member 206 or a separate tube, as desired. In other embodiments, the struts 224 and one or more of the proximal and distal collars 226, 228 may be formed as separate structures that are coupled together. For example, the struts 224 may be welded, brazed, soldered, adhered, etc. to the proximal and/or distal collars 226, 228. Other coupling techniques may be used, as desired. It is contemplated that the coupling mechanism may be dependent, at least in part, on the material of the struts 224 and/or the collars 226, 228. When the expandable basket 220 is a separate component from the inner tubular member 206, at least one of the proximal collar 226 or the distal collar 228 may be fixedly secured to an outer surface of the inner tubular member 206 while the other of the proximal collar 226 or the distal collar 228 is movably disposed over the inner tubular member 206 to allow the basket 220 to radially expand. In one example, the proximal collar 226 may be fixedly secured to the inner tubular member 206 while the distal collar 228 may be slidably disposed over the inner tubular member 206. In such a configuration, the distal collar 228 may move distally to collapse the expandable basket 220 and move proximally to expand the expandable basket 220. The reverse configuration is also contemplated in which the distal collar 228 may be fixedly secured to the inner tubular member 206 while the proximal collar 226 may be slidably disposed over the inner tubular member 206.

The expandable basket 220 may be self-expandable or may require an external force to expand from a collapsed state. Self-expandable members may be formed of any material or structure that is in a compressed state when force is applied and in an expanded state when force is released. Such members may be formed, for example, of shape memory alloys such as nitinol or any other self-expandable materials. When employing such shape-memory materials, the expandable basket 220 may be heat set in the expanded state and then compressed to fit within the outer tubular member 204, for example. In another embodiment, a spring may be provided to effect expansion. It is contemplated that nickel-titanium alloys may enable kink-resistant folding and self-expansion. In other examples, magnetic alloys, metals, metal alloys, polymers, composites, etc. may be used to form the expandable basket 220.

In other instances, a manual force applied to the inner tubular member 206 may manipulate or actuate the expandable basket 220 between the expanded and collapsed state. For example, an actuation element may include a central wire or rod that extends through the expandable basket 220 and is coupled to the distal collar 228. Alternatively, external forces such as, but not limited to, pneumatic methods, compressed fluid, pull wires, push wires, rods, or the like may also be employed to expand the expandable basket 220. According to this embodiment, a pulling force exerted proximally on the wire which may cause the struts 224 to expand and move the expandable basket 220 into an expanded state. A pushing force exerted distally on the wire may elongate the struts 224 and/or otherwise shift the expandable basket 220 to a compressed or elongated state. Other actuation mechanisms may also be utilized. For example, the guidewire 216 may be used to exert a proximal pushing force on a distal end of the distal collar 226. In such an instance, the guidewire 216 may include an enlarged region having a diameter greater than a diameter of the lumen of the tissue collection device 202 such that proximal actuation of the guidewire 216 causes the enlarged region of the guidewire 216 to contact the distal collar 226 and further proximal actuation of the guidewire 216 causes the distal collar 226 to move proximally and expand the expandable basket 220. It is contemplated that manual actuation of the expandable basket 220 using a wire, rod, etc., may put a higher force on the expandable basket 220 which may allow for greater engagement of the basket with a target tissue.

When the tissue collection device 202 is disposed within the outer tubular member 204, the expandable basket 220 may be restrained in a compressed reduced diameter or delivery configuration by the outer tubular member 204 surrounding the tissue collection device 202. In other examples, the expandable basket 220 may assume a collapsed configuration until an actuation force is applied to the expandable basket 220. In the collapsed configuration, the tissue collection device 202 may have a smaller diameter than the expanded deployed configuration. The distal end region 210 of the outer tubular member 204 may be positioned such that the outer tubular member 204 surrounds and covers the length of the tissue collection device 202 during delivery. The outer tubular member 204 may have sufficient hoop strength to retain the tissue collection device 202 in its reduced diameter state.

The tissue collection system 200 may be advanced through the body towards the target location, as desired. The tissue collection system 200 may be advanced with or without the use of a guidewire 216. Once the tissue collection device 202 is positioned adjacent to the target region, the restraining forces maintaining the tissue collection device 202 in the radially compressed configuration may be removed to deploy the tissue collection device 202.

The tissue collection device 202 may be deployed by actuating the second handle, for example, by distally pushing the second handle, while maintaining the first handle in a fixed position. Thus, the inner tubular member 206 may be distally advanced relative to the outer tubular member 204. In other words, the inner tubular member 206 may be distally advanced while the outer tubular member 204 is held stationary. The reverse configuration is also contemplated. For example, the outer tubular member 204 may be proximally retracted while the inner tubular member 206 is held stationary. As the inner tubular member 206 is distally advanced, the biasing force is removed from the exterior of the tissue collection device 202 and the expandable basket 220 may assume its radially expanded, unbiased, deployed configuration, shown in FIG. 4. Alternatively, the expandable basket 220 may remain collapsed until an actuation force is applied to the expandable basket 220 (e.g., using a pull-wire or other actuation mechanism) to move the expandable basket 220 to the radially expanded, deployed configuration.

In the radially expanded configuration, the struts 224 may flex radially outwards such that a proximal end region and a distal end region of the expandable basket 220 extend at a non-parallel angle relative to a longitudinal axis of the expandable basket 220 and an intermediate region extends approximately parallel to the longitudinal axis of the expandable basket 220. However, the expandable basket 220 may take other shapes in the expanded configuration, as desired. The expandable basket 220 may be sized and shaped such that when the expandable basket 220 is in the expanded configuration, the struts 224 may interface with a collection site. In some cases, the expandable basket 220 may be expanded until at least a portion of the struts 224 contact the collection site. It is contemplated that in some cases, the expandable basket 220 may not fully expand before contacting the collection site.

Once the tissue collection device 202 is deployed from the outer tubular member 204 and the expandable basket 220 is expanded, the second handle may be actuated to repeatedly distally advance, proximally retract, and/or rotate the tissue collection device 202 along the target collection site. This may cause the struts 224 to brush against the tissue surface to dislodge and capture cells. It is contemplated that the edges 230a, 230b of the struts 224 may be angled or sharpened to scratch the collection site. In the illustrated embodiment, each edge of the plurality of struts 224 is not labeled for brevity and ease of understanding. In some examples, the struts 224 may further include texturing, such as, but not limited to, a roughed or grainy surface, on an outer surface and/or inner surface thereof. The texturing may be configured to release and capture cells from the stricture. The texturing may be macro-texturing or micro-texturing, as desired. It yet other examples, the struts 224 may include barbs. The barbs may extend radially inward or radially outwards from the struts 224 and may be configured to further disrupt the collection site to release cells for collection. The barbs may be similar in form and function to the barbs 122 shown and described with respect to FIG. 3. It is further contemplated that one or more of the struts 224 may include other tissue engaging features, such as, but not limited to, bristles, anywhere along a length of the struts 224.

The struts 224 may further include one or more teeth 232a-c (collectively, 232) extending from one or more of the struts 224. The teeth 232 may be formed as a single monolithic structure with the struts 224. For example, the struts 224 and teeth 232 may be formed from a cut tube with the struts 224 and teeth 232 formed by cutting regions from the tube. The teeth 232 may be coupled to the struts 224 at a first end thereof and extend to a free end. The free end may be pointed to engage tissue at the collection site. However, this is not required. In some embodiments, the free end may be rounded or take another form, as desired. While each strut 224 is illustrated each including three teeth 232, it is contemplated that the struts 224 may include fewer than three or more than three teeth 232, as desired. In some examples, one or more of the struts 224 may be free from teeth 232. It is further contemplated that the struts 224 may have differing numbers of teeth 232 from one another. Further, while the teeth 232 are shown as being located in an intermediate region of the struts 224, the teeth 232 may be positioned anywhere along a length of the struts 224, as desired.

The tissue collection device 202 may further include one or more optional tissue capturing mechanisms (not explicitly shown). The tissue capturing mechanism may include a brush or membrane that may collect or trap cells that have been dislodged by the expandable basket 220. Some illustrative brushes are described in commonly assigned U.S. Patent Application No. 63/309,8208, titled TISSUE SAMPLE DEVICE AND METHODS, the disclosure of which is hereby incorporated by reference. It is contemplated that the tissue capturing mechanism may be positioned proximal to the expandable basket, distal to the expandable basket 220, or anywhere along a length of the expandable basket 220, as desired.

Once the clinician has captured cells from the target site, the inner tubular member 206 may be proximally retracted until the tissue collection device 202 is disposed within the lumen 208 of the outer tubular member 204. When the expandable basket 220 is self-expanding, the proximal retraction of the inner tubular member 206 may cause the basket 220 to collapse as the basket 220 is drawn into the lumen 208 of the outer tubular member 204. When expandable basket 220 is manually expanded, the basket 220 may be moved into the collapsed configuration before proximally retracting the inner tubular member 206. In some embodiments, the inner tubular member 206 may include one or windows or cut-out regions 234 for trapping cells or tissues therein as the expandable basket 220 is collapsed. The cut-out regions 234 may be generally aligned with tissue collection device 202 so that as the tissue collection device 202 is collapsed or retracted, the collapsed tissue collection device 202 draws cells or tissue into the cut-out regions 234. In some embodiments, the teeth 232 may also rest within the cut-out regions 234. It is contemplated that the guidewire 216 may be removed from the lumen 214 of the inner tubular member 206 prior to collapsing the expandable basket 220 to allow more tissue or cells to be pulled into the cut-out regions 234.

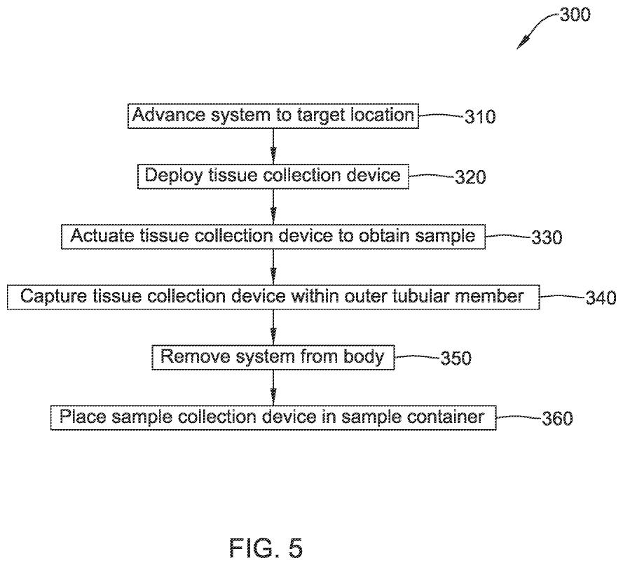

FIG. 5 is an illustrative flow chart of a method 300 for collecting a tissue sample using the systems 10, 200 and/or tissue collection devices 12, 100, 202 of FIGS. 1-4. To begin, the tissue collection system 10, 200 may be advanced to the target location in the body, as shown at block 310. In some embodiments, the tissue collection system 10, 200 may be advanced through the working channel an endoscope, a distal end of which may be positioned near the target location. For example, to obtain a sample from the common bile duct, an endoscope may be advanced through the esophagus, through the stomach, and into the duodenum. The tissue collection system 10, 200 may be positioned within the endoscope as it is being positioned or may be subsequently advanced therethrough. The guidewire 42, 216 of the tissue collection system 10, 200 may then be distally advanced to cannulate the common bile duct. The inner tubular member 16, 206 may then be distally advanced over the guidewire to deploy the tissue collection device 12, 100, 202, as shown at block 320. As described above, the inner tubular member 16, 206 may be distally advanced while the outer tubular member 14, 204 is stationary such that the tissue collection device 12, 100, 202 exits the outer tubular member 14, 204. It is contemplated that when the guidewire 42, 216 is centrally located within the tissue collection system 10, 200, the inner tubular member 16, 206 may not be biased to one side as with a tissue collection system which advances a brush through a separate channel from the guidewire. The co-axial arrangement of the guidewire 42, 216 and the inner tubular member 16, 206 may allow the tissue collection device to pass the ampulla, bends, and stricture with ease. In other examples, the tissue collection system 10, 200 and/or tissue collection devices 12, 100, 202 may be attached to a side of endoscope.

Once the tissue collection device 12, 100, 202 has been positioned at the target location, the inner tubular member 16, 206 may be actuated (e.g., using the second handle 30) back and forth (e.g., proximally and distally) to drag the struts 46, 18, 208, 224 along the collection site to gather cells, as shown at block 330. In some cases, the inner tubular member 16, 206 may be rotated in addition to or alternatively to proximal and distal movement. Once the sample has been collected, the inner tubular member 16, 206 may be proximally retracted to draw the tissue collection device 12, 100, 202 back into the lumen 18, 208 of the outer tubular member 14, 204, as shown at block 340. The tissue collection system 10, 200 may be removed from the body, as shown at block 350. The inner tubular member 16, 206 may then be cut (e.g., using wire cutters or other cutting device) at a location proximal to the tissue collection device 12, 100, 202 such that the tissue collection device 12, 100, 202 may be placed into a sample container, as shown at block 360.

The materials that can be used for the various components of the medical device system 10, 200 (and/or other systems disclosed herein) and the various elements thereof disclosed herein may include those commonly associated with medical devices. For simplicity purposes, the following discussion refers to the tissue collection system 10, 200 and/or the tissue collection device 12, 100, 202. However, this is not intended to limit the devices and methods described herein, as the discussion may be applied to other elements, members, components, or devices disclosed herein, such as, but not limited to, the inner and outer tubular members 16, 206, 14, 204, handles 24, 30, guidewire 42, 216, etc. and/or elements or components thereof.

In some embodiments, the tissue collection system 10, 200 and/or the tissue collection device 12, 100, 202, and/or components thereof, may be made from a metal, metal alloy, polymer (some examples of which are disclosed below), a metal-polymer composite, ceramics, combinations thereof, and the like, or other suitable material.

Some examples of suitable polymers may include polytetrafluoroethylene (PTFE), ethylene tetrafluoroethylene (ETFE), fluorinated ethylene propylene (FEP), polyoxymethylene (POM, for example, DELRIN® available from DuPont), polyether block ester, polyurethane (for example, Polyurethane 85A), polypropylene (PP), polyvinylchloride (PVC), polyether-ester (for example, ARNITEL® available from DSM Plastics), ether or ester based copolymers (for example, butylene/poly(alkylene ether) phthalate and/or other polyester elastomers such as HYTREL® available from DuPont), polyamide (for example, DURETHAN® available from Bayer or CRISTAMID® available from Elf Atochem), elastomeric polyamides, block polyamide/ethers, polyether block amide (PEBA, for example available under the trade name PEBAX®), ethylene vinyl acetate copolymers (EVA), silicones, polyethylene (PE), MARLEX® high-density polyethylene, Marl MARLEX®ex low-density polyethylene, linear low density polyethylene (for example REXELL®), polyester, polybutylene terephthalate (PBT), polyethylene terephthalate (PET), polytrimethylene terephthalate, polyethylene naphthalate (PEN), polyetheretherketone (PEEK), polyimide (PI), polyetherimide (PEI), polyphenylene sulfide (PPS), polyphenylene oxide (PPO), poly paraphenylene terephthalamide (for example, KEVLAR®), polysulfone, nylon, nylon-12 (such as GRILAMID® available from EMS American Grilon), perfluoro(propyl vinyl ether) (PFA), ethylene vinyl alcohol, polyolefin, polystyrene, epoxy, polyvinylidene chloride (PVdC), poly(styrene-b-isobutylene-b-styrene) (for example, SIBS and/or SIBS 50A), polycarbonates, ionomers, biocompatible polymers, other suitable materials, or mixtures, combinations, copolymers thereof, polymer/metal composites, and the like.

Some examples of suitable metals and metal alloys include stainless steel, such as 304V, 304L, and 316LV stainless steel; mild steel; nickel-titanium alloy such as linear-elastic and/or super-elastic nitinol; other nickel alloys such as nickel-chromium-molybdenum alloys (e.g., UNS: N06625 such as INCONEL® 625, UNS: N06022 such as HASTELLOY® C-22®, UNS: N10276 such as HASTELLOY® C276®, other HASTELLOY® alloys, and the like), nickel-copper alloys (e.g., UNS: N04400 such as MONEL® 400, NICKELVAC® 400, NICORROS® 400, and the like), nickel-cobalt-chromium-molybdenum alloys (e.g., UNS: R30035 such as MP35-N® and the like), nickel-molybdenum alloys (e.g., UNS: N10665 such as HASTELLOY® ALLOY B2®), other nickel-chromium alloys, other nickel-molybdenum alloys, other nickel-cobalt alloys, other nickel-iron alloys, other nickel-copper alloys, other nickel-tungsten or tungsten alloys, and the like; cobalt-chromium alloys; cobalt-chromium-molybdenum alloys (e.g., UNS: R30003 such as ELGILOY®, PHYNOX®, and the like); platinum enriched stainless steel; titanium; combinations thereof; and the like; or any other suitable material.

As alluded to herein, within the family of commercially available nickel-titanium or nitinol alloys, is a category designated “linear elastic” or “non-super-elastic” which, although may be similar in chemistry to conventional shape memory and super elastic varieties, may exhibit distinct and useful mechanical properties. Linear elastic and/or non-super-elastic nitinol may be distinguished from super elastic nitinol in that the linear elastic and/or non-super-elastic nitinol does not display a substantial “super-elastic plateau” or “flag region” in its stress/strain curve like super elastic nitinol does. Instead, in the linear elastic and/or non-super-elastic nitinol, as recoverable strain increases, the stress continues to increase in a substantially linear, or a somewhat, but not necessarily entirely linear relationship until plastic deformation begins or at least in a relationship that is more linear than the super elastic plateau and/or flag region that may be seen with super elastic nitinol. Thus, for the purposes of this disclosure linear elastic and/or non-super-elastic nitinol may also be termed “substantially” linear elastic and/or non-super-elastic nitinol.

In some cases, linear elastic and/or non-super-elastic nitinol may also be distinguishable from super elastic nitinol in that linear elastic and/or non-super-elastic nitinol may accept up to about 2-5% strain while remaining substantially elastic (e.g., before plastically deforming) whereas super elastic nitinol may accept up to about 8% strain before plastically deforming. Both of these materials can be distinguished from other linear elastic materials such as stainless steel (that can also be distinguished based on its composition), which may accept only about 0.2 to 0.44 percent strain before plastically deforming.

In some embodiments, the linear elastic and/or non-super-elastic nickel-titanium alloy is an alloy that does not show any martensite/austenite phase changes that are detectable by differential scanning calorimetry (DSC) and dynamic metal thermal analysis (DMTA) analysis over a large temperature range. For example, in some embodiments, there may be no martensite/austenite phase changes detectable by DSC and DMTA analysis in the range of about −60 degrees Celsius (° C) to about 120° C. in the linear elastic and/or non-super-elastic nickel-titanium alloy. The mechanical bending properties of such material may therefore be generally inert to the effect of temperature over this range of temperature. In some embodiments, the mechanical bending properties of the linear elastic and/or non-super-elastic nickel-titanium alloy at ambient or room temperature are substantially the same as the mechanical properties at body temperature, for example, in that they do not display a super-elastic plateau and/or flag region. In other words, across a broad temperature range, the linear elastic and/or non-super-elastic nickel-titanium alloy maintains its linear elastic and/or non-super-elastic characteristics and/or properties.

In some embodiments, the linear elastic and/or non-super-elastic nickel-titanium alloy may be in the range of about 50 to about 60 weight percent nickel, with the remainder being essentially titanium. In some embodiments, the composition is in the range of about 54 to about 57 weight percent nickel. One example of a suitable nickel-titanium alloy is FHP-NT alloy commercially available from Furukawa Techno Material Co. of Kanagawa, Japan. Other suitable materials may include ULTANIUM™ (available from Neo-Metrics) and GUM METAL™ (available from Toyota). In some other embodiments, a super-elastic alloy, for example a super-elastic nitinol can be used to achieve desired properties.

In at least some embodiments, portions or all of the tissue collection system 10, 200 and/or the tissue collection device 12, 100, 202, and/or components thereof, may also be doped with, made of, or otherwise include a radiopaque material. Radiopaque materials are understood to be materials capable of producing a relatively bright image on a fluoroscopy screen or another imaging technique during a medical procedure. This relatively bright image aids the user of the tissue collection system 10, 200 and/or the tissue collection device 12, 100, 202 in determining its location. Some examples of radiopaque materials can include, but are not limited to, gold, platinum, palladium, tantalum, tungsten alloy, polymer material loaded with a radiopaque filler, and the like. Additionally, other radiopaque marker bands and/or coils may also be incorporated into the design of the tissue collection system 10, 200 and/or the tissue collection device 12, 100, 202 to achieve the same result.

In some embodiments, a degree of Magnetic Resonance Imaging (MRI) compatibility is imparted into the tissue collection system 10, 200 and/or the tissue collection device 12, 100, 202. For example, the tissue collection system 10, 200 and/or the tissue collection device 12, 100, 202, and/or components or portions thereof, may be made of a material that does not substantially distort the image and create substantial artifacts (e.g., gaps in the image). Certain ferromagnetic materials, for example, may not be suitable because they may create artifacts in an MRI image. The tissue collection system 10, 200 and/or the tissue collection device 12, 100, 202, or portions thereof, may also be made from a material that the MRI machine can image. Some materials that exhibit these characteristics include, for example, tungsten, cobalt-chromium-molybdenum alloys (e.g., UNS: R30003 such as ELGILOY®, PHYNOX®, and the like), nickel-cobalt-chromium-molybdenum alloys (e.g., UNS: R30035 such as MP35-N® and the like), nitinol, and the like, and others.

In some embodiments, an exterior surface of the medical device system 10 (including, for example, an exterior surface of the delivery system) may be sandblasted, beadblasted, sodium bicarbonate-blasted, electropolished, etc. In these as well as in some other embodiments, a coating, for example a lubricious, a hydrophilic, a protective, or other type of coating may be applied over portions or all of the outer sheath, or in embodiments without an outer sheath over portions of the delivery system, or other portions of the medical device system 10. Hydrophobic coatings such as fluoropolymers provide a dry lubricity which improves device handling and device exchanges. Lubricious coatings improve steerability and improve lesion crossing capability. Suitable lubricious polymers are well known in the art and may include silicone and the like, hydrophilic polymers such as high-density polyethylene (HDPE), polytetrafluoroethylene (PTFE), polyarylene oxides, polyvinylpyrrolidones, polyvinyl alcohols, hydroxy alkyl cellulosics, algins, saccharides, caprolactones, and the like, and mixtures and combinations thereof. Hydrophilic polymers may be blended among themselves or with formulated amounts of water insoluble compounds (including some polymers) to yield coatings with suitable lubricity, bonding, and solubility.

The coating and/or sheath may be formed, for example, by coating, extrusion, co-extrusion, interrupted layer co-extrusion (ILC), or fusing several segments end-to-end. The layer may have a uniform stiffness or a gradual reduction in stiffness from the proximal end to the distal end thereof. The gradual reduction in stiffness may be continuous as by ILC or may be stepped as by fusing together separate extruded tubular segments. The outer layer may be impregnated with a radiopaque filler material to facilitate radiographic visualization. Those skilled in the art will recognize that these materials can vary widely without deviating from the scope of the present disclosure.

It should be understood that this disclosure is, in many respects, only illustrative. Changes may be made in details, particularly in matters of shape, size, and arrangement of steps without exceeding the scope of the disclosure. This may include, to the extent that it is appropriate, the use of any of the features of one example embodiment being used in other embodiments. The disclosure's scope is, of course, defined in the language in which the appended claims are expressed.

What is claimed is:

1. A tissue collection system, the system comprising:
an outer tubular member having a proximal end region and a distal end region and defining a lumen extending from the proximal end region to the distal end region;
an inner tubular member slidably disposed within the lumen of the outer tubular member, the inner tubular member defining a lumen extending from a proximal end region to a distal end region of the inner tubular member; and
a tissue collection device disposed adjacent to the distal end region of the inner tubular member, the tissue collection device comprising an expandable basket;
wherein the tissue collection device is movable between a retracted delivery position and an extended sample collection position.

2. The tissue collection system of claim 1, wherein the expandable basket comprises a plurality of longitudinally extending struts.

3. The tissue collection system of claim 2, wherein one or more edges of at least one strut of the plurality of struts has a sharpened surface.

4. The tissue collection system of claim 2, wherein a surface of at least one strut of the plurality of struts is textured.

5. The tissue collection system of claim 2, further comprising one or more barbs coupled to at least one strut of the plurality of struts.

6. The tissue collection system of claim 5, wherein the one or more barbs comprises a wire wound around the at least one strut.

7. The tissue collection system of claim 6, wherein at least one free end of the wire extends radially from the at least one strut.

8. The tissue collection system of claim 2, further comprising one or more teeth extending from at least one strut.

9. The tissue collection system of claim 8, wherein the one or more teeth are formed as a single monolithic structure with the at least one strut.

10. The tissue collection system of claim 2, wherein the plurality of struts comprises a plurality of individual filaments, the filaments coupled to one another at their respective proximal and distal ends.

11. The tissue collection system of claim 1, wherein the tissue collection device comprises a cut tube.

12. The tissue collection system of claim 1, wherein the tissue collection device is a monolithic structure with the inner tubular member.

13. The tissue collection system of claim 1, wherein the tissue collection device is coupled to the inner tubular member.

14. The tissue collection system of claim 1, wherein the tissue collection device is self-expanding.

15. The tissue collection system of claim 1, further comprising an actuation mechanism coupled to a proximal or distal end of the tissue collection device.

16. The tissue collection system of claim 1, wherein the inner tubular member further comprises one or more cut-out regions, the one or more cut-out regions adjacent to the tissue collection device.

17. A tissue collection system, the system comprising:
an outer tubular member having a proximal end region and a distal end region and defining a lumen extending from the proximal end region to the distal end region;
an inner tubular member slidably disposed within the lumen of the outer tubular member, the inner tubular member defining a lumen extending from a proximal end region to a distal end region of the inner tubular member; and
a tissue collection device disposed adjacent to the distal end region of the inner tubular member, the tissue collection device comprising a radially expandable basket having a plurality of longitudinally extending struts extending between a proximal collar and a distal collar;
wherein the tissue collection device is movable between a retracted collapsed delivery position and an extended expanded sample collection position.

18. The tissue collection system of claim 17, wherein the tissue collection device is a cut tube.

19. The tissue collection system of claim 17, wherein at least one strut of the plurality of struts includes a radially extending tissue disruption feature.

20. A tissue collection system, the system comprising:
an outer tubular member having a proximal end region and a distal end region and defining a lumen extending from the proximal end region to the distal end region;
an inner tubular member slidably disposed within the lumen of the outer tubular member, the inner tubular member defining a lumen extending from a proximal end region to a distal end region of the inner tubular member;
a tissue collection device disposed adjacent to the distal end region of the inner tubular member, the tissue collection device comprising a radially expandable basket having a plurality of longitudinally extending wires extending between a proximal collar and a distal collar; and
at least one barb coupled to at least one wire of the plurality of longitudinally extending wires, the at least one barb including a radially extending free end;
wherein the tissue collection device is movable between a retracted collapsed delivery position and an extended expanded sample collection position.
